(19) United States
(12) Patent Application Publication
(10) Pub. No.: US 2016/0136201 A1
(43) Pub. Date: May 19, 2016

(54) METHOD OF MEDICALLY TREATING EBOLA AND OTHER ORGANISMS

(71) Applicant: Ross A. Deardorff, Indianapolis, IN (US)

(72) Inventor: Ross A. Deardorff, Indianapolis, IN (US)

(21) Appl. No.: 14/945,311

(22) Filed: Nov. 18, 2015

Related U.S. Application Data
(60) Provisional application No. 62/081,190, filed on Nov. 18, 2014.

(51) Int. Cl. A61K 33/20; A61K 33/00; A61M 1/28; A61K 9/00

(52) U.S. Cl. CPC A61K 33/20; A61K 9/0031; A61K 9/0014; A61K 33/00; A61M 1/287

(57) ABSTRACT

A method of treating Ebola in a human patient includes covering sensitive body parts of the patent, and immersing the patient in a bath of sodium hypochlorite solution. A concentration of the sodium hypochlorite in the solution being high enough that the solution penetrates skin of the patient to react with fat tissue of the skin.

### CROSS-REFERENCE TO RELATED APPLICATIONS

This application claims the benefit of U.S. Provisional Application No. 62/081,190, filed on Nov. 18, 2014, which the disclosure of which is hereby incorporated by reference in its entirety for all purposes.

### BACKGROUND

U.S. Pat. No. 6,589,568 to Camper et al. discloses a therapeutic body lotion containing alakali metal hypohalite.

### SUMMARY OF THE INVENTION

The invention comprises, in one form thereof, a method of treating Ebola in a human patient, including covering sensitive body parts of the patient, and immersing the patient in a bath of sodium hypochlorite solution. A concentration of the sodium hypochlorite in the solution with chemical or electrical enhancers being high enough that the solution penetrates skin of the patient to react with fat tissue of the skin.

The invention comprises, in another form thereof, a method of killing Ebola and other organisms in a human patient. The method includes preparing a sodium hypochlorite solution, and introducing the sodium hypochlorite solution into a body cavity of the patient via peritoneal dialysis or other dialysis.

The invention comprises, in yet another form thereof, a method of killing Ebola and other organisms in a human patient. The method includes preparing a sodium hypochlorite solution. The sodium hypochlorite solution is introduced into a body of the patient via an enema.

The invention comprises, in still another form thereof, a method of killing Ebola and other organisms in a human patient. The method includes preparing a sodium hypochlorite solution. The sodium hypochlorite solution is introduced into the oral or nasal cavity of the patient via a swab stick.

### DETAILED DESCRIPTION

The embodiments hereinafter disclosed are not intended to be exhaustive or limit the invention to the precise forms disclosed in the following description. Rather the embodiments are chosen and described so that others skilled in the art may utilize its teachings.

The invention may provide a method of treating a patient infected with Ebola or other organisms with a sodium hypochlorite solution, such as Dakin's Solution®. A patient with Ebola or other virus, fungus, or bacterial microorganism may take a bath in sodium hypochlorite, so that the solution penetrates the skin at a deep level, possibly reaching the internal organs and body cavity, thereby killing the offending microorganism.

The method could also be used to help heal chronic wounds. The method of delivery could be in the form of a patch, with extended release of the sodium hypochlorite active ingredient.

Sodium hypochlorite reacts with the fat in a person's skin, reducing the fat, and making it possible to the sodium hypochlorite to penetrate the body to a deeper level or greater quantity than if the sodium hypochlorite is applied topically. Repeated baths in sodium hypochlorite solution may lead to weight loss, due to the reduction in the patient's fat. In one embodiment, the patient wears a belt that is pre-soaked in the sodium hypochlorite solution, in order to focus the solution on the patient's mid-section, thereby reducing the fat localized in the stomach area.

The concentration of sodium hypochlorite used could be anywhere approximately between 0.001% and 0.50%, and may be monitored and regulated. The treatment protocol may be monitored by a healthcare professional, and may be modified as necessary, depending upon a variety of factors, such as the type and severity of wound, condition, disease, or microorganism; the length of time the patient has had the condition; any known allergies or adverse reactions the patient has to chlorine or sodium hypochlorite; the size/body mass index of the patient; and the results achieved after a few treatments.

Instead of a bath, a patient may take a shower in the sodium hypochlorite solution. Medical personnel treating a patient with Ebola or other virus, disease, etc. may have their body and/or clothes showered in the sodium hypochlorite solution before and/or after treatment of the patient.

Regardless of the type of treatment, discontinuing the treatment may allow the skin to naturally repair itself.

The solution may be packaged in various sizes, depending upon the application, from swab sticks, one ounce bottles or packets to 330 gallon totes.

Under proper manufacturing techniques, sodium hypochlorite can be used to, or can be allowed to, compromise the patient's skin in order to allow more sodium hypochlorite to enter the body for therapeutic treatments. Sodium hypochlorite solution can also be used in peritoneal applications for systemic treatment of Ebola and other microorganisms, while following strict controls.

The sodium hypochlorite solution may be used to aid medical personnel who must wear personal protective equipment (PPE) following contact with a patient with a hazardous or infectious disease or condition. The healthcare professional can soak or shower themselves or their PPE with sodium hypochlorite solution. In specific embodiments, the healthcare professional can soak or shower themselves or their PPE with sodium hypochlorite solutions in the form of products manufactured by Century Pharmaceuticals, Inc. in Indianapolis, Indiana, including Dakin's Solution® Full Strength, Dakin's Solution® Half Strength, Dakin's Solution® Quarter Strength, Di-Dak-Sol® (Diluted Dakin's Solution), or WoundClenz™ OTC. The sodium hypochlorite solution could be sprayed on the PPE worn by medical personnel, and particularly on the joints or juncture points in the equipment, prior to removal of the equipment.

This cleaning process may be performed in controlled conditions. For example, the person's clothes may be washed in a containment area, with or without a shower water rinse. The run-off water may be retained in a large storage tank for treatment with additional sodium hypochlorite solution in order to kill any remaining microorganisms prior to releasing the run-off into a public sewer system. Anything that accidentally comes into contact with the PPE may also be sprayed with one of the Dakin's products.

Century Pharmaceuticals may manufacture its Dakin's Solution® products using commercial sodium hypochlorite 12%-18% solution diluted with purified water to a concentration of 0.001%-0.50% and buffered with sodium bicarbonate to a pH of approximately 10. The sodium hydroxide within the sodium hypochlorite product reacts with the fat in the skin, breaking the fat down, and allowing more chlorine to enter the patient's system than if the sodium hypochlorite were manufactured without sodium hydroxide or other chemical or electrical enhancers. A concentration of the sodium hydroxide in the sodium hypochlorite solution may be such that the sodium hydroxide reacts with and breaks down the fat tissue in the skin. Other chemical or electrical enhancers may also be used to allow penetration into the skin for therapeutic effect.

The invention may be applied to any pathogen. In one embodiment, the sodium hypochlorite solution is used topically only. The sodium hypochlorite solution may be in the form of Dakin's Solution®, using a strength approximately between 0.001% and 0.5%, depending upon the method of application.

The reason for applying the sodium hypochlorite solution drug systemically is twofold. First, at high concentrations oral use can be toxic and ineffective as the stomach will change the pH of the drug and destroy the drug. Secondly, the passage of sodium hypochlorite may cause damage to the mouth, esophagus, and stomach.

In a first inventive method, the patient, with sensitive body parts covered and protected, is placed into a tub with a solution of sodium hypochlorite. The sensitive body parts (e.g., eyes, genitals) may be covered with waterproof tape, waterproof shorts, waterproof briefs, or goggles, for example. The strength of the solution, and the time duration of the bath, may be determined by a qualified care giver. Three criteria may be called for. First, the solution may be strong enough to permit penetration of the skin for a therapeutic dose. Second, the strength of the solution should be low enough to be safe for the patient, and not cause damage. Third, the solution may be strong enough to react with the skin to increase the penetration of the sodium hypochlorite with the skin fat tissue. This may be called for to cause the skin to “open” and allow the sodium hypochlorite to penetrate the body. This third criterion may lead to effective results. The skin repair may take place naturally upon discontinuation of the Sodium Hypochlorite treatment.

In a second inventive method, the patient may be treated with sodium hypochlorite solution (strength and the time duration of treatment to be determined by care giver) via peritoneal dialysis. This method may introduce the sodium hypochlorite solution into the body cavity. The sodium hypochlorite solution remaining in the body after treatment and body interaction may be flushed out of the body with a cleaning solution, such as a saline solution, for example.

In a third inventive method, the sodium hypochlorite solution may be administered to the patient via an enema (strength and the time duration of treatment to be determined by care giver). In this situation, the pH of the solution and of the intestinal tract are alkaline, making the two environments compatible.

While this invention has been described as having an exemplary design, the present invention may be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

What is claimed is:

1. A method of treating Ebola in a human patient, the method comprising:
covering sensitive body parts of the patient; and
at least partially immersing the patient in a bath of sodium hypochlorite solution, a concentration of the sodium hypochlorite in the solution being high enough that the solution penetrates skin of the patient to react with fat tissue of the skin.

2. The method of claim 1 wherein a concentration of sodium hypochlorite in the sodium hypochlorite solution is approximately between 0.001% and 0.50%.

3. The method of claim 2 wherein a concentration of sodium hypochlorite in the sodium hypochlorite solution is approximately between 0.0125% and 0.50%.

4. The method of claim 1 wherein the sensitive body parts of the patent are covered with tape, waterproof clothing, and/or goggles.

5. The method of claim 1 wherein a concentration of sodium hypochlorite in the sodium hypochlorite solution, and a time duration of the patient being at least partially immersed in the bath, are such that the sodium hypochlorite penetrates the skin and reaches an internal organ of the patient and/or a body cavity of the patient.

6. The method of claim 1 wherein the sodium hypochlorite solution is buffered with sodium bicarbonate to a pH of approximately 10.

7. The method of claim 6 wherein the sodium hypochlorite solution includes sodium hydroxide, and a concentration of the sodium hydroxide is such that the sodium hydroxide reacts with and breaks down the fat tissue in the skin.

8. A method of killing Ebola and other organisms in a human patient, the method comprising:
preparing a sodium hypochlorite solution; and
introducing the sodium hypochlorite solution into a body cavity of the patient via dialysis.

9. The method of claim 8 further comprising flushing out remaining sodium hypochlorite solution in the body cavity of the patient with a cleaning solution.

10. The method of claim 9 wherein the remaining sodium hypochlorite solution in the body cavity of the patient is flushed out via dialysis.

11. The method of claim 8 wherein a concentration of the sodium hypochlorite in the sodium hypochlorite solution is approximately between 0.001% and 0.50%.

12. The method of claim 11 wherein a concentration of sodium hypochlorite in the sodium hypochlorite solution is approximately between 0.0125% and 0.50%.

13. The method of claim 8 wherein the sodium hypochlorite solution comprises a first sodium hypochlorite solution, the method further comprising topically applying a second sodium hypochlorite solution to the patient before or after the bath, the second sodium hypochlorite solution including sodium hydroxide.

14. The method of claim 13 wherein the second sodium hypochlorite solution is applied to the patient via a bath, a lotion applied to skin of the patient, a patch adhered to skin of the patient, a swab, or a belt worn by the patient.

15. A method of killing Ebola and other organisms in a human patient, the method comprising:
preparing a sodium hypochlorite solution, a pH of the solution being alkaline; and
introducing the sodium hypochlorite solution into a body of the patient via an enema.

16. The method of claim 15 further comprising flushing out a remainder of the sodium hypochlorite solution in the body of the patient with a cleaning solution via an enema.

17. The method of claim 15 wherein a concentration of the sodium hypochlorite in the sodium hypochlorite solution is approximately between 0.001% and 0.50%.

18. The method of claim 17 wherein a concentration of sodium hypochlorite in the sodium hypochlorite solution is approximately between 0.0125% and 0.50%.

19. The method of claim 15 wherein the sodium hypochlorite solution comprises a first sodium hypochlorite solution, the method further comprising topically applying a second sodium hypochlorite solution to the patient before or after the bath, the second sodium hypochlorite solution including sodium hydroxide.

20. The method of claim 19 wherein the second sodium hypochlorite solution is applied to the patient via a bath, a lotion applied to skin of the patient, a patch adhered to skin of the patient, a swab, or a belt worn by the patient.
